(19) United States
(12) Patent Application Publication
(10) Pub. No.: US 2026/0114811 A1
(43) Pub. Date: Apr. 30, 2026

(54) METHODS FOR ANATOMICAL LOCATION ASSIGNMENT OF SENSOR-ENABLED SPINAL IMPLANTS

(71) Applicant: Warsaw Orthopedic, Inc., Warsaw, IN (US)

(72) Inventors: Jerald L. Redmond, Germantown, TN (US); Newton H. Metcalf, JR., Memphis, TN (US); Mark C. Dace, Collierville, TN (US); Elias Mulugeta Ayana, Blaine, MN (US); Adam D. Glaser, Collierville, TN (US); Arjun Siby Kurian, Chesterfield, MO (US)

(21) Appl. No.: 19/347,486

(22) Filed: Oct. 1, 2025

Related U.S. Application Data
(60) Provisional application No. 63/711,183, filed on Oct. 24, 2024.

(51) Int. Cl. A61B 5/00

(52) U.S. Cl. CPC A61B 5/6847; A61B 5/0013; A61B 5/0031; A61B 5/4566; A61B 5/7425; A61B 5/7475

(57) ABSTRACT

Devices and methods for configuring a telemetry system are disclosed. A method includes displaying a representation of an anatomical structure of a subject, the representation including multiple potential implant positions, each potential implant position being user-selectable. The method further includes receiving a user selection of one of the potential implant positions. The method further includes receiving a distinguishing identifier of a selected implant. The method further includes, in response to receiving an indication that the selected implant has been installed, associating the distinguishing identifier of the selected implant with the user-selected implant position. The method further includes, in response to receiving physical location information related to the installed implant, associating the installed implant with the received location information.

### CROSS-REFERENCE TO RELATED APPLICATION

This application claims the benefit of and priority to U.S. Provisional Patent Application No. 63/711,183 filed Oct. 24, 2024, the entire disclosure of which is incorporated by reference herein.

The present disclosure generally relates to mechanical and electrical sensor assemblies for implant devices, and more particularly to implants that transmit sensor data to a remote reader device.

### BACKGROUND

Treatment of spinal disorders, such as degenerative disc disease, disc herniations, scoliosis or other curvature abnormalities, fractures, and so forth often require surgical treatments. For example, spinal fusion may be used to limit motion between vertebral members. As another example, implants may be used to preserve motion between vertebral members.

Surgical treatment typically involves the use of longitudinal members, such as spinal rods. Longitudinal members may be attached to the exterior of two or more vertebral members to assist with the treatment of a spinal disorder. Longitudinal members may provide a stable, rigid column that helps bones to fuse, and may redirect forces over a wider area away from a damaged or defective region. Also, rigid longitudinal members may help in spinal alignment.

Screw assemblies may be used to connect a longitudinal member to a vertebral member. A screw assembly may include a pedicle screw, hook, or other connector, among other components. A pedicle screw can be placed in, above and/or below vertebral members that were fused, and a longitudinal member can be used to connect the pedicle screws and inhibit or control movement. A set screw can be used to secure the connection of a longitudinal member to a pedicle screw, hook or other connector.

Some implants include sensors configured to measure forces between components, temperature or other indication of infection at a single surgical site, and/or indications of post-operative progress. As such “smart” implants become more prevalent, the volume of data from a single surgical site will only increase. Furthermore, it will only become more difficult to interpret the cacophony of data, particularly if there is any ambiguity about where each “smart” sensor is located (and, thus, what its data relates to). Therefore, there is a need to reliably and accurately associate “smart” sensor location with the anatomical structure where it is installed.

This document describes methods and systems that are directed to addressing the problems described above, and/or other issues.

### SUMMARY

The techniques of this disclosure generally relate to telemetry systems that include “smart” implants and techniques for configuring such systems. Issues associated with prior solutions are addressed by the subject matter of the independent claims included in this document. Additional advantageous aspects are included in the dependent claims.

In one aspect, the present disclosure provides a telemetry system. The telemetry system includes one or more implants, each implant including a sensor configured to obtain data related to a subject, a distinguishing identifier, and a transmitter configured to transmit the distinguishing identifier and the obtained data. The telemetry system further includes a telemetry configuration system. The telemetry configuration system is configured to display a representation of an anatomical structure of the subject, the representation including multiple potential implant positions, each potential implant position being user-selectable. The telemetry configuration system is further configured to receive a user selection of one of the potential implant positions and receive the distinguishing identifier of a selected implant of the one or more implants. In response to receiving an indication that the selected implant has been installed, the telemetry configuration system is configured to associate the distinguishing identifier with the user-selected implant position. In response to receiving physical location information related to the installed implant, the telemetry configuration system is configured to associate the installed implant with the received location information.

Implementations of the disclosure may include one or more of the following optional features. In some examples, the telemetry system further includes a remote reader having a display device, the remote reader configured to receive the distinguishing identifier and the data obtained from the implants and display the obtained data on the display device. The telemetry configuration system may be configured to associate the selected implant with the user-selected implant position by associating the selected implant with the user-selected implant position in a data store accessible to the remote reader. The telemetry configuration system may be configured to receive the distinguishing identifier of the selected implant by wirelessly receiving a unique identifier. The telemetry configuration system may be configured to receive the distinguishing identifier of the selected implant by receiving, via a camera, a machine-readable code associated with the selected implant. In some examples, receiving the indication that the selected implant has been installed includes receiving sensor data from the selected implant and recognizing that the received sensor data includes a signature that the selected implant has been installed. The representation of the anatomical structure of the subject may include a medical image of the subject. Receiving the physical location information related to the installed implant may include receiving the physical location information from a surgical navigation system. Receiving the physical location information related to the installed implant may include analyzing a medical image to determine the physical location information.

In some examples, the telemetry configuration system is further configured to, after receiving the user selection of one of the potential implant positions, receive user annotation related to the user-selected implant position and associate the user annotation with the user-selected implant position. The telemetry configuration system may be configured to receive the physical location information related to the installed implant by receiving tracking information associated with a tool in contact with the installed implant. Receiving the physical location information related to the installed implant may include receiving a medical image of the subject and receiving a location of the installed implant in the medical image. The telemetry system may further include a surgical planning system, wherein the telemetry configuration system is configured to receive the distinguishing identifier of the selected implant from the surgical planning system.

In another aspect, the present disclosure provides a method of configuring a telemetry system. The method includes displaying a representation of an anatomical structure of a subject, the representation including multiple potential implant positions, each potential implant position being user-selectable. The method further includes receiving a user selection of one of the potential implant positions and receiving a distinguishing identifier of a selected implant. The method further includes, in response to receiving an indication that the selected implant has been installed, associating the distinguishing identifier with the user-selected implant position. The method further includes, in response to receiving physical location information related to the installed implant, associating the installed implant with the received location information.

Implementations of the disclosure may include one or more of the following optional features. In some examples, the method further includes, after receiving the user selection of one of the potential implant positions, receiving a user annotation related to the user-selected implant position and associating the user annotation with the user-selected implant position. Receiving the distinguishing identifier of the selected implant may include wirelessly receiving a unique identifier from the selected implant. Receiving the indication that the selected implant has been installed may include receiving sensor data from the selected implant and recognizing that the received sensor data includes a signature that the selected implant has been installed. Receiving the physical location information related to the installed implant may include analyzing a medical image to determine the physical location information. In some examples, the method further includes receiving a pre-operative medical image of the subject. Displaying the representation of the anatomical structure of the subject may include displaying the received medical image of the subject. Receiving the indication that the selected implant has been installed may include receiving the indication from a robotic navigation system.

### BRIEF DESCRIPTION OF THE DRAWINGS

The accompanying drawings are incorporated into this document and form a part of the specification.

FIG. 1 illustrates an example telemetry system.

FIG. 2 shows an example environment for installing implants.

FIGS. 3A, 3B, 4A, and 4B show example user interfaces.

FIG. 5A shows an example environment for installing implants

FIG. 5B illustrates an example of activating an implant.

FIG. 6 shows a flowchart of a method of configuring a telemetry system.

FIG. 7 shows a block diagram of an example of internal hardware that may be used to contain or implement program instructions according to an embodiment.

In the drawings, like reference numbers generally indicate identical or similar elements. Additionally, generally, the left-most digit(s) of a reference number identifies the drawing in which the reference number first appears.

### DETAILED DESCRIPTION

Sensor-enabled spinal implants will provide the ability for remote monitoring of patients following surgery to evaluate progression towards recovery. Spinal fusion procedures can involve the use of multiple implants, including pedicle screws, hooks, set screws, rods, cages, rod connectors, tether connectors, cross-links, plates, and interspinous fixation devices. For instance, six pedicle screws are typically placed for a two-level construct, whereas 18 screws could be placed for an eight-level construct, including use of two pedicle screws and two set screws at each level, dual or quad rods, and multiple cages across different levels for a complex adult procedure. With use of sensor-enabled implants, constructs could involve placement of both standard implants and sensor-enabled implants. Accurately and reliably keeping track of the locations where sensor-enabled implants have been placed is important for interpretating measurement data with remote monitoring. The systems and methods of this disclosure enhance and streamline the process of installing sensor-enabled implants and provide for automatically recording the physical positions of the implants during installation for increased post-surgical data fidelity and understanding.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting.

In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior.” Generally, similar spatial references of different aspects or components indicate similar spatial orientation and/or positioning, i.e., that each “first end” is situated on or directed towards the same end of the device.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

The following discussion includes a description of a telemetry system and methods of configuring the telemetry system in accordance with the principles of the present disclosure. Reference is made in detail to the exemplary embodiments of the present disclosure, which are illustrated in the accompanying figures.

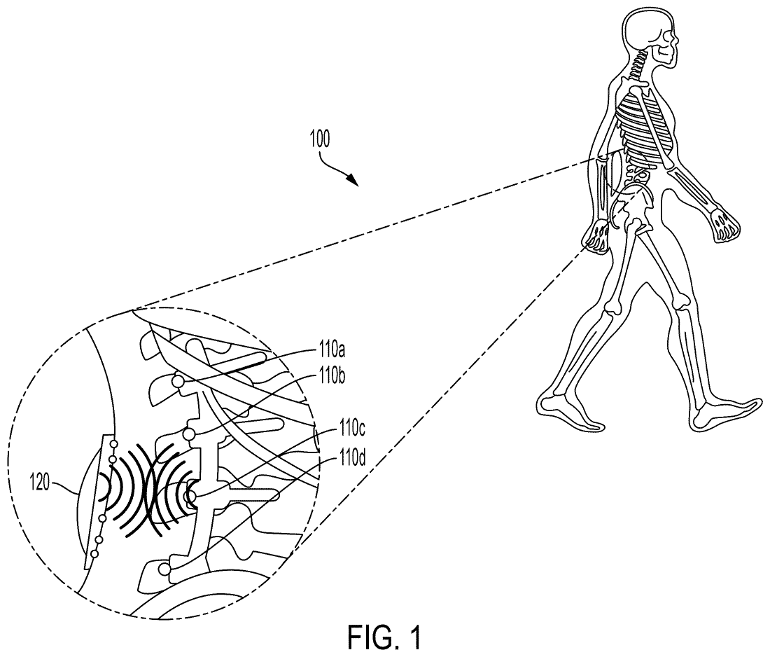

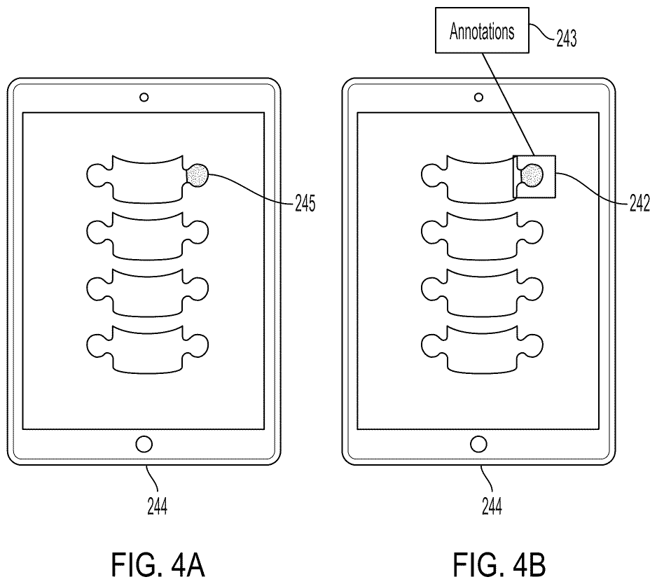

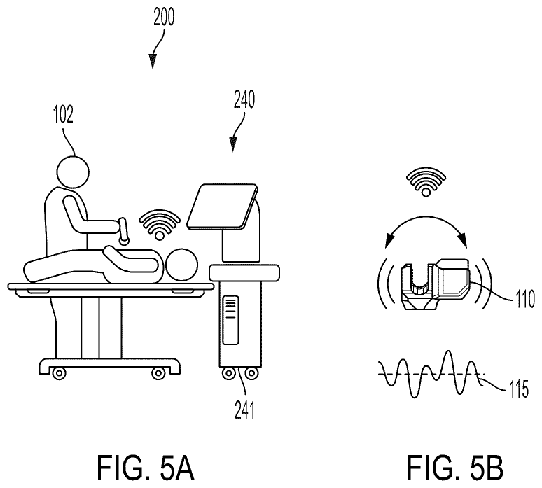

Referring to FIG. 1, an example telemetry system 100 is shown. For example, the telemetry system 100 may be a surgical-site load monitoring system (using one or more strain gauges or other force sensors) and/or an infection-monitoring system (using one or more temperature sensors). FIG. 1 illustrates a spinal-fusion construct having multiple separate sensor-equipped implants 110a-d, each of which may have one or more sensors. Sensors may include, without limitation, force or strain sensors, position sensors, gyroscopes, accelerometers, temperature sensors, and so forth. Other embodiments within the scope of this disclosure include multiple implant systems, e.g., multiple spinal-fusion constructs, and/or individual sensor-equipped implants 110. In some examples, only a fraction of the implants are configured to measure and transmit data. Other combinations and permutations of implant systems and/or sensor-equipped implants 110 are also within the scope of the disclosure. In addition to the one or more sensors, each sensor-equipped implant 110 also includes a transmitter configured to transmit the sensor data to an external reader device 120. FIG. 1 shows the reader 120 disposed on or near the lower back of a subject. In other examples, the reader device 120 may be disposed elsewhere on the subject, or the reader device 120 may be disposed at a location that is remote from the subject, such as a bed-side monitor. Furthermore, the reader device 120 may be integrated into a surgical planning system, a robotic navigation system (241, FIG. 5A), or other system configured to assist in treatment of the subject. Each sensor-equipped implant 110 may also include a power source, such as a battery (e.g., either primary or rechargeable) and associated electronics to enable the implant 110 to obtain and transmit the sensor data (e.g., a microprocessor, transceiver, antenna, and so forth).

In one or more embodiments, the telemetry system 100 includes an external reader device 120 configured to receive sensor data from the implants 110. For the embodiments in which the telemetry system 100 includes an array of implants 110 having various sensors, the received data from the one or more sensors may be compared to one another to diagnose the quality of the surgical procedure, the integrity of the implant 110, and/or an infection at the surgical site, among other functions.

In some examples, each implant 110 is secured/anchored to a bone of a subject 120. For example, the implant 110 may include a screw configured to anchor the implant 110 to a pedicle of the subject's vertebra and/or another component of a spinal construct, such as a hook member, a cross-link connector, an offset connector, or a hybrid hook-screw member, etc.

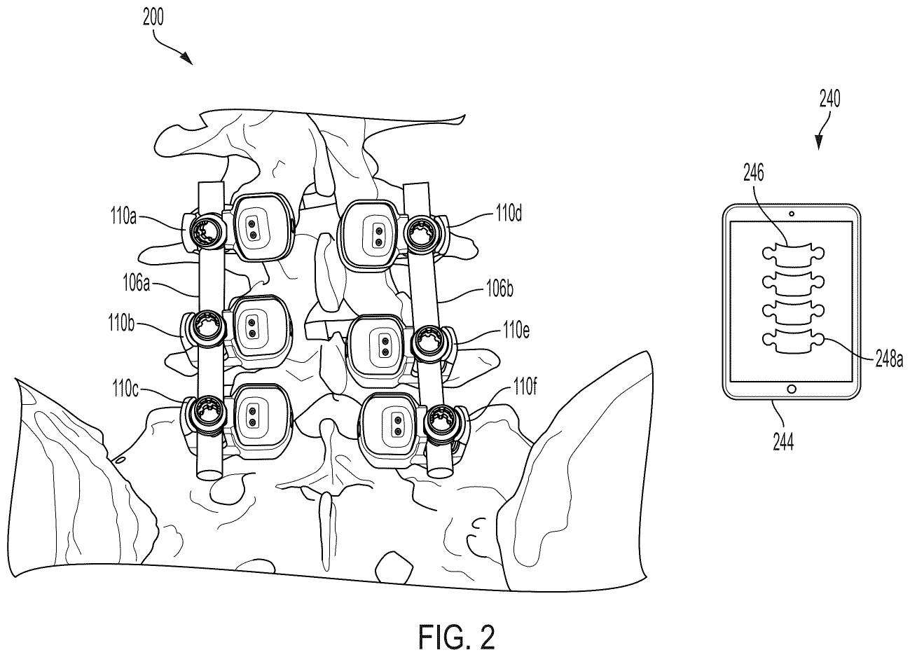

The implant 110 may also be configured to receive and secure a longitudinal member 106 (FIG. 2) such as a spinal rod. The longitudinal member 106 may be used to connect adjacent implants 110, each of which may be secured to a subject's bone (each, adjacent vertebrae) and a spinal rod 106, also secured within a respective implant 110. Implants 110 may have additional uses, including, but not limited to constraining vertebral motion using a tether or ligament tape.

One or more external readers 120 is configured to receive data from the “smart” (sensor-equipped) implants 110. The reader(s) 120 may display the data, interpret the data, and/or retransmit the data to one or more other reader devices 120. In order to interpret and/or display sensor data in a meaningful way, the reader devices 120 may also receive (e.g., from a configuration data store), configuration information related to the implants 110. For example, the configuration data may include a position of each sensor-equipped implant 110 within the spinal construct. The position information may include an associated spine level, e.g., a region, such as Cervical (C), Thoracic (T), Lumbar (L), Sacral(S) and Coccygeal (Cx), and a numerical level within the region. The position information may also indicate which side of the spine the implant 110 is installed on, e.g., left or right. The configuration information may also include the physical location of the implant 110, e.g., relative to a reference location of the subject's anatomy. The reader device(s) 120 may apply the configuration information to the sensor data to detect an anomaly or to evaluate progression towards recovery. If equipped with a display, the reader device(s) 120 may also use the configuration data when displaying the sensor data, e.g., to color-code or otherwise label the source of the data and/or to show the data relative to the position of the implant 110. Alternatively, a reader device 120 may transmit information to a separate device for display, such as another computer, a mobile device, tablet, or the like. Showing, e.g., temperature data relative to the position of temperature sensors in the construct may help a medical professional 102 (FIG. 5A) pinpoint the scope of a post-operative infection. Similarly, showing force information relative to the position of each implant 110 may help the medical professional 102 detect portions of the construct that are not functioning as expected.

Each implant 110 may have an associated identifier that distinguishes the implant 110 from other implants 110. For example, each implant 110 may have a unique serial number or a Global Trade Item Number (GTIN). The identifier may be printed on the implant 110, e.g., in plain text and/or in a machine-readable form, such as a barcode or QR code. In some examples, each implant 110 transmits its distinguishing identifier in addition to transmitting sensor data, so that the reader device 120 is able to associate the sensor data with a particular implant 110.

FIG. 2 shows an example environment 200 for installing implants 110 (with or without sensors). A surgeon or other medical professional 102 may install a construct including multiple implants 110a-110f and one or more longitudinal members 106a, 106b to add extra support and strength the subject's spine, and/or prevent movement of vertebrae, e.g., to allow a spinal fusion to heal. The surgeon 102 may also use the construct to maintain or correct anatomic alignment of spinal segments by distributing the loads acting on the spine. In these and other cases, the surgeon 102 may want to monitor information such as the forces applied by the longitudinal member(s) 106 to the implants 110, position and/or orientation of each implant 110, temperature data, and/or other sensor data. For example, an abrupt change in forces applied to an implant 110 may indicate a bone fracture or a malfunction of the construct, such as loosening or dislodgement or one or more implants 110. Even in the absence of a malfunction, the surgeon 102 may want to monitor, e.g., fusion status against the surgeon's expectations. For these and other examples, a load-sensing implant 110 may be configured to transmit sensor data to the external reader device 120 intra-operatively and/or post-operatively.

The environment 200 also includes a telemetry configuration system 240. The medical professional 102 (or someone acting on behalf of the medical professional 102) may interact with the telemetry configuration system 240 to configure the telemetry system 100, e.g., providing input as the construct is installed (or prior to installation). The telemetry configuration system 240 also provides information to the medical professional 102, e.g., to facilitate the configuration and/or installation. That is, the telemetry configuration system 240 may provide bidirectional communication with the medical professional 102. In some examples, the telemetry configuration system 240 includes a dedicated display screen 244. In some examples, the telemetry configuration system 240 may be integrated with or otherwise be associated with a surgical planning and/or a software-guided surgical platform. In some examples, the telemetry configuration system 240 includes an app for a mobile device, such as a tablet or smart phone, and/or is accessible from the mobile device, such that the mobile device acts as the display screen 244 and/or input device for the telemetry configuration system 240. As shown, the display screen 244 of the telemetry configuration system 240 is a mobile device.

The telemetry configuration system 240 is configured to reliably and accurately associate each “smart” sensor location with the anatomical structure where it is installed. As shown in FIG. 2, the telemetry configuration system 240 displays a representation 246 of the subject's spine. The representation 246 may be pre-annotated to indicate potential positions (e.g., 248a) where implants 110 may be installed. The annotation 243 may be in any suitable form to indicate the available positions, including color-coded and/or user-selectable regions of the representation 246, an associated text-based list of available positions, etc. In examples involving other types of procedures, the telemetry configuration system 240 may display representations 246 of other anatomical structures, such as limb or joint bones, etc. and may be pre-annotated to indicate potential implant positions for those procedures.

In some examples, the representation 246 is a 2D or 3D medical image of the actual subject's anatomy. Example medical images include pre-operative x-ray images, CT images, ultrasound images, MRI image “slices,” intra-operative fluoroscope images, and the like. One or more pre-operative images may be used for surgical planning. That is, the surgeon 102 may include pre-operative information, e.g., from one or more medical images, in the telemetry configuration system 240 (and/or in a surgical planning system). For example, the surgeon 102 may, based on pre-operative information, provide annotations for the telemetry configuration system 240 to associate with specific potential-implant locations. In some examples, the telemetry configuration system 240 (and/or a dedicated surgical-planning system) may automatically provide annotations. For example, a surgical-planning system may automatically segment an image of the patient's spine into separate vertebrae. The surgical-planning system may further identify and/or label vertebrae levels in the image and/or potential positions for implants. In some examples, the surgical-planning system includes a machine-learning module that has been trained to identify vertebrae levels in medical images, e.g., using supervised machine learning. In other examples, semi-supervised, unsupervised, and self-supervised techniques may be used as well.

In other examples, the representation 246 is a drawing or artistic illustration, a computer rendering, a synoptic display, or other non-medical image. The representation 246 may simply be a text listing of available implant positions without departing from the scope of the invention. Regardless of the form of the representation 246, the telemetry configuration system 240 is further configured to receive user selection of a location where an implant 110 will be installed. The user selection may be made, e.g., using a touch-sensitive area of the display screen, or by any other suitable input method, including (but not limited to) keyboard, mouse or other pointing device, microphone, and the like that provides sufficient information for the telemetry configuration system 240 to understand the user's selection.

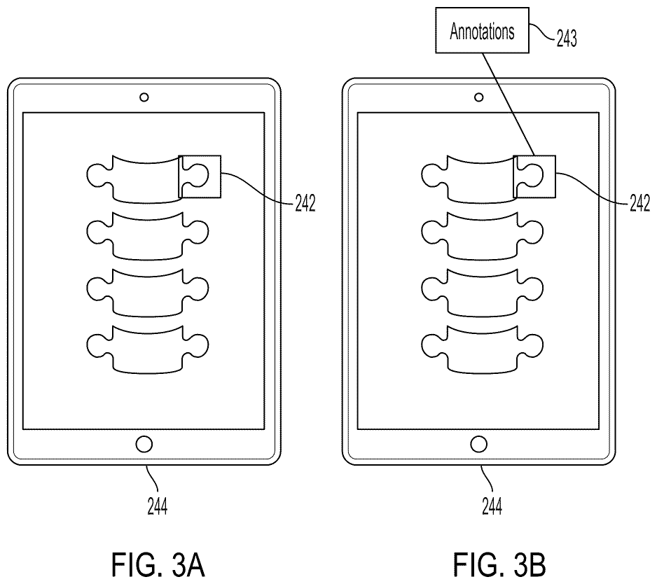

Referring to FIG. 3A, the user (e.g., the surgeon 102 or medical professional 102) has selected one potential position, the right side of the upper vertebra in the display 735. In response, the telemetry configuration system 240 provides an indication 242 of the user selection. As shown, the indication 242 is a square that surrounds the selected position. In other examples, the selection may be indicated using a different symbol and/or color coding and may be accompanied with an audible indication 242 that the user selection has been received. Referring to FIG. 3B, once the user selection has been received, the telemetry configuration system 240 may provide for user annotation of the selected position, e.g., by selecting a menu option, right-clicking using a mouse or other pointer, etc. The selection and annotation may be performed pre-operatively, intra-operatively and/or post-operatively, allowing the user to include information as it becomes available during these phases. Similar to the user selection, the telemetry configuration system 240 may provide for user annotation using, e.g., a keyboard, touch-sensitive screen, microphone, camera, etc. In some examples, the telemetry configuration system 240 displays the annotation 243 or otherwise makes the annotation 243 acceptable to the user for revision, addition, and/or deletion. For example, if a potential position has one or more associated annotations 243, the potential position may always display the annotation 243 or may display the annotation 243 whenever the user selects the potential position, or whenever the user hovers over the position. In some examples, the telemetry configuration system 240 displays one or more icons to indicate the presence of user annotation. The telemetry configuration system 240 may display a different icon for each type of annotation, e.g., text, image, audio, and so forth. In some examples, the telemetry configuration system 240 makes the full annotation available in response to user selection of the icon.

While a potential position is selected (e.g., while the indication 242 is displayed), the telemetry configuration system 240 may also provide for receiving the distinguishing identifier of an implant 110 to be installed at the selected position. The telemetry configuration system 240 may receive the distinguishing identifier using a keyboard, camera, or microphone. For example, a serial number may be manually entered using a keyboard, or spoken into a microphone and interpreted, e.g., using a speech-to-text algorithm. Using a camera, the telemetry configuration system 240 may be configured to read text, barcodes, QR codes, or other machine-readable formats. In some examples, the telemetry configuration system 240 uses machine vision, image processing, and/or a trained machine-learning model to recognize the type (e.g., make/model) of the implant 110. In some examples, the telemetry configuration system receives the distinguishing identifier wirelessly, e.g., using Radio Frequency Identification (RFID), a wireless protocol such as Bluetooth Low Energy, or other form of communication. Referring now to FIG. 4A, after the telemetry configuration system 240 receives the distinguishing identifier, the telemetry configuration system 240 may provide an indication 245 that an implant 110 (and its distinguishing identifier) has been associated with the position. As shown in FIG. 4A, the indication may be a filled circle or other indication that is visually different than the indication 242 of user selection. Referring now to FIG. 4B, even after the telemetry configuration system 240 has associated the implant 110 with the position, the telemetry configuration system 240 may provide for ongoing selection and/or annotation. For example, the telemetry configuration system 240 may provide for deleting or updating an association.

In some examples, the telemetry configuration system 240 receives information including one or more potential implant positions and associated distinguishing identifiers. That is, the steps of manually entering this information in the telemetry configuration system 240 may be replaced by importing this information from a surgical-planning system or other source. For example, a surgical-planning system may include annotated medical images which indicate the location where each implant is to be installed and the distinguishing identifier for each implant. The surgical-planning system may include additional information, including additional annotation, and/or the actual physical location of each implant location (e.g., with respect to a reference position of the subject's anatomy). The telemetry configuration system 240 may receive all this information from the surgical-planning system.

In some examples, the telemetry configuration system 240 receives the distinguishing identifier from the implant 110. As disclosed above, the telemetry configuration system 240 may receive the distinguishing identifier using a camera. Furthermore, the telemetry configuration system 240 may receive the distinguishing identifier wirelessly from the implant 110. In some examples, the telemetry configuration system 240 receives the distinguishing identifier from each implant 110 as each implant is installed. FIG. 5A shows an example environment 200 for installing implants 110, including a telemetry configuration system 240. As shown, the telemetry configuration system 240 is integrated into a robotic navigation system 241. In other examples, the telemetry configuration system 240 may be integrated into an image-guidance system, a computer-assisted surgery system, or the like). The robotic navigation system 241 is configured to track the position of one or more surgical tools used during a procedure. The robotic navigation system 241 may also include one or more cameras and/or other imaging systems. In some examples, the robotic navigation system 241 receives the distinguishing identifier using the cameras or imaging systems. The robotic navigation system 241 may also receive the distinguishing identifier wirelessly from each implant 110.

Furthermore, the robotic navigation system 241 may provide for selecting a potential implant position. For example, the medical professional 102 may select a potential implant position using a surgical tool that is tracked by the robotic navigation system 241. The robotic navigation system 241 may use the tracked position and/or images of the surgical site to identify the selected implant position. In some examples, the medical professional 102 triggers the robotic navigation system 241 to accept the position selection through the surgical tool, using a control, such as a button or a triggering motion of the surgical tool, such as tapping the tool near the selected implant position. In other examples, the medical professional 102 may trigger an individual implant 110, causing the implant 110 to transmit an indication that it has been triggered. The indication may be received by the reader device 120 (either stand-alone or, e.g., incorporated into the robotic navigation system 241). If the reader device 120 is stand-alone, it may relay the indication to the robotic navigation system 241 directly or, e.g., via a cloud server), causing the robotic navigation system 241 to accept the position selection.

In some examples, as the medical professional 102 installs each implant 110, the medical professional 102 also activates the implant 110. That is, the medical professional 102 may cause the implant 110 to begin transmitted sensor data and to transmit its associated distinguishing identifier. The robotic navigation system 241 may receive the transmitted sensor data and the distinguishing identifier and associate the distinguishing identifier with the currently selected potential implant position.

In some examples, each implant 110 is associated with a particular position in advance, i.e., prior to installation. In these examples, the robotic navigation system 241 may be used to verify that the implant 110 is in its associated position. For example, the medical professional 102 may use a surgical tool to simultaneously select an implant location and activate the implant to begin transmitting. The robotic navigation system 241 may receive the selected position and may receive the associated distinguishing identifier from the implant 110. The robotic navigation system 241 may indicate whether the pre-defined association is accurate. If not, the robotic navigation system 241 may prompt the medical professional 102 to update the association or exchange the implant 110. In a similar fashion, when the implants 110 are associated with particular positions during the procedure, e.g., as they are installed, the robotic navigation system 241 may be used to perform a final confirmation that the associations are correct.

Referring now to FIG. 5B, the telemetry configuration system 240 (e.g., robotic navigation system 241) may be configured to recognize characteristic sensor data from an implant 110. For example, the sensor data may include accelerometer data 115. The telemetry configuration system 240 may be configured to recognize accelerometer data 115 that indicates that the implant 110 has been tapped by a surgical instrument or that the head of an implant 110 has been snapped onto a pedicle screw, or that the head of an implant 110 has been manipulated (e.g., moved side-to-side in a characteristic way), and so forth. The telemetry configuration system 240 may analyze the received accelerometer data 115 to recognize the characteristic signature of an implant 110 that has been installed and/or intentionally selected by the medical professional 102. In response, the telemetry configuration system 240 may associate the implant 110 with the most recently selected potential position. The medical professional 102 may be prompted to accept or reject the association (e.g., to accept the association unless rejected within a threshold period of time). The telemetry configuration system 240 may be configured to recognize other characteristic sensor data including, without limitation, temperature or force data, which may also act as a signature of the implant 110 being selected.

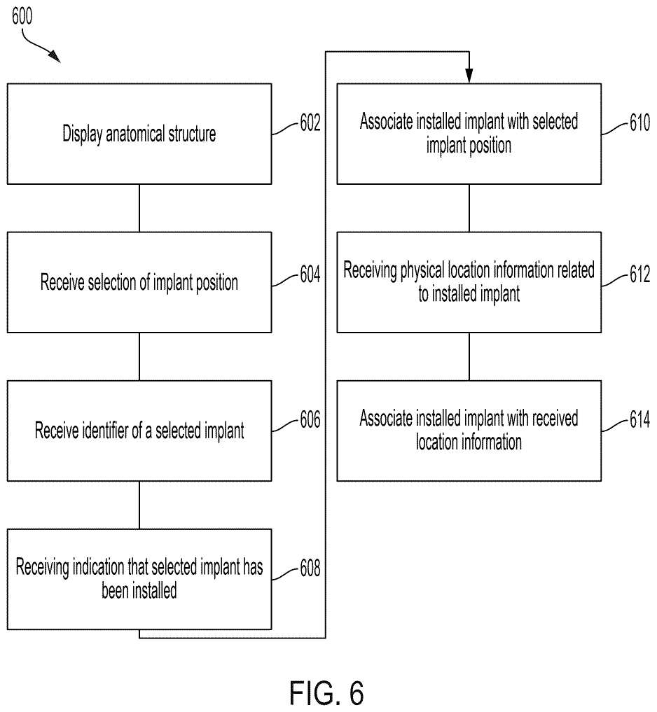

FIG. 6 shows a flowchart 600 of a method of configuring a telemetry system. At step 602, the method includes displaying a representation of an anatomical structure of a subject. As disclosed above, the representation may be a drawing or artistic illustration, a medical image, or other form that also includes a representation of potential positions for implants 110 to be installed and/or attached to the anatomical structure. At step 604, the method includes receiving a user selection of one of the potential implant positions. That is, the method includes receiving an indication of a particular implant position to be used for subsequent operations. The subsequent operations include annotating the selected position and/or associating a particular implant 110 with the selected position. The form of the indication may be related to the form of the representation of the anatomical structure. For example, if the representation is an artistic illustration rendered on a touch-sensitive display screen, the user may indicate a particular implant position by touching the display screen at or near the rendered position. Alternatively, if the representation is merely an enumerated list of available positions within the anatomical structure, the user may indicate a particular implant position by referencing a particular item in the list. Other modes of selection are also within the scope of the disclosure.

In some examples, the method also includes associating the implant location with a medical image of the patient's anatomy. The medical image may be a pre-operative or intra-operative image of the patient's anatomy. In some embodiments, the medical professional 102 examines the medical image and identifies the implant location in the image. In some examples, the medical image is displayed on a display screen, e.g., of a surgical-planning system or a robotic navigation system. In this case, the medical professional 102 (or other personnel) may associate each available implant position with its corresponding position in the medical image. In some examples, a surgical-planning system, computer-assisted surgery system, and/or robotic navigation system automatically associates potential implant locations with corresponding positions in the medical image. For example, a surgical-planning system may automatically segment an image of the patient's spine into separate vertebrae, identify vertebrae levels, and/or identify left/right locations at each vertebra before associating potential implant locations with each identified left/right location. The surgical-planning system may include one or more machine-learning models trained to identify vertebrae, levels, and/or left/right positions in medical images. A computer-assisted surgery system and/or robotic navigation system may register the medical image of the subject to the physical position of the subject intraoperatively using semi-automated methods (e.g., landmark registration, CT/Fluoro registration) or automated methods (automatic image registration with dynamic referencing, camera-based computer vision, electromagnetic-based registration). This registration enables the medical professional to localize the position of a tracked instrument with respect to the subject's anatomy for associating the physical implant location with the user-selected or planned implant location.

At step 606, the method includes receiving a distinguishing identifier of a selected implant 110. In some examples, receiving the distinguishing identifier includes receiving a serial number or similar unique identifier applied to the implant 110. In some examples, the distinguishing identifier is displayed on a label, tag, or other printed material associated with the implant 110. In some examples, receiving the distinguishing identifier includes receiving a wireless transmission from the implant 110, where the wireless transmission includes a unique identifier. The unique identifier may be a network address or other identifier associated with a wireless transmission protocol. At step 608, the method includes receiving an indication that the selected implant 110 has been installed. In some examples, the medical professional 102 who installed the implant 110 also provides the indication that the selected implant 110 has been installed. For example, the medical professional 102 may select a menu item, checkbox, or other graphical input widget to indicate that the implant 110 has been installed. Alternatively, the medical professional 102 may utter a recognizable word or phrase, such as “installed.” In some examples, the medical professional 102 operates a physical control, such as a button or pedal and/or manipulates a surgical tool to indicate that the implant 110 has been installed.

In some examples, receiving the indication that the selected implant 110 has been installed includes receiving an image of the implant 110 in the installed position (e.g., using a camera or a medical imaging system). In some examples, receiving the indication that the selected implant 110 has been installed includes receiving a transmission from the installed implant 110 indicating that the implant 110 has been installed. In some examples, the received transmission includes characteristic sensor data indication that the implant 110 has been installed. In response to receiving the indication that the selected implant 110 has been installed, the method further includes, at step 610, associating the installed implant 110 with the implant position selected in step 604.

At step 612, the method includes receiving physical location information related to the installed implant 110. In some examples, receiving the physical location information related to the installed implant 110 includes receiving physical location information from a surgical and/or robotic navigation system. That is, the navigation system may be configured to track the physical location of a tool, which the medical professional 102 places in contact with the implant 110. In other examples, receiving physical location information related to the installed implant 110 includes receiving one or more images of the implant 110 in its installed position. That is, the navigation system may be configured to precisely track the location of anatomical features, e.g., based on x-ray or other medical images. The navigation system may provide the physical location of each implant 110 with respect to other implants and/or with respect to a reference position of the subject's anatomy.

In some examples, implants 100 use a short-range wireless communication protocol, such as RFID or BlueTooth. In these examples, the physical location of the implant may be determined based on signals produced by the implants 110. The physical location of the implants may be determined, e.g., based on the relative strength of the signals produced by the implants 110 measured at multiple different locations around the implant 110. In some examples, the external reader measures signal strength from the implants 110 for use in determining the physical location of each implant 110. In some examples, a medical professional 102 interacts with one or more medical images to identify each implant and associate the implant with a particular position and/or a distinguishing identifier.

In some examples, the navigation system automatically identifies each implant 110 in medical images and automatically associates each identified implant with a particular position and/or a distinguishing identifier. For example, a computer-assisted surgery system (CAS) and/or robotic navigation system may be configured to receive image data (e.g., from one or more cameras configured to capture images of the surgical site). The CAS system and the telemetry configuration system 240 may cooperate to associate the identified implants 110 with their respective distinguishing identifier. In some examples, the surgical assistant system provides the particular position of each identified implant (along with, e.g., its spinal level and left/right location) to the telemetry configuration system 240 to associate each implant 110 with its distinguishing identifier. In some examples, a robotic navigation system may cooperate with the telemetry configuration system 240 to determine a physical location of each implant 110. For example, the robotic navigation system may track the position of a surgical tool and provide the tracked position to the telemetry configuration system 240. The telemetry configuration system 240 may prompt the medical professional 102 to use the tracked tool to locate each installed implant (e.g., in a specified order). As each tracked location is provided to the telemetry configuration system 240, the telemetry configuration system 240 may associate the physical location with the corresponding implant 110. In some examples, the telemetry configuration system 240 is integrated with a robotic navigation system and/or a surgical planning/assistant system.

In response to receiving the physical location information, the method includes, at step 614, associating the physical location information with the installed implant 110. For example, the physical location information may be associated with the implant's distinguishing identifier. In some examples, the implant position selected in step 604 and the physical location received in step 612 may both be associated with the distinguishing identifier received in step 606. Furthermore, other information related to the implant 110, such as the implant's complement of sensors, associated calibration data, etc. may also be associated with the distinguishing identifier, such that all this data is available to inform subsequent interpretation and display of sensor data by the one or more reader devices 120. For example, the data may be stored in a data store accessible to the remote reader 120.

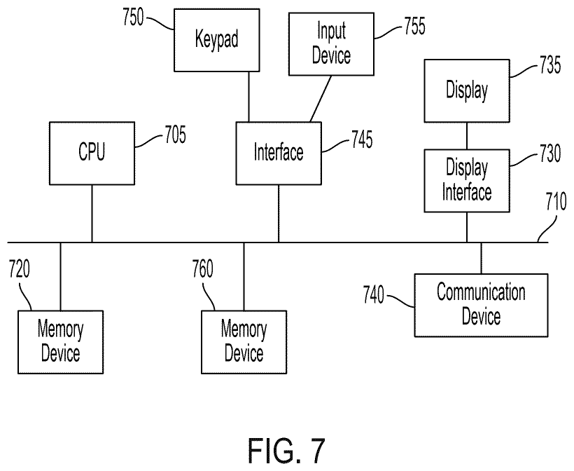

FIG. 7 illustrates example hardware that may be used to contain or implement program instructions. A bus 710 serves as the main information highway interconnecting the other illustrated components of the hardware. Central Processing Unit (CPU) 705 is the central processing unit of the system, performing calculations and logic operations required to execute a program. CPU 705, alone or in conjunction with one or more of the other elements disclosed in FIG. 7, is an example of a processor as such term is used within this disclosure. Read only memory (ROM) and random-access memory (RAM) constitute examples of non-transitory computer-readable storage media 720, memory devices or data stores as such terms are used within this disclosure.

Program instructions, software or interactive modules for providing the interface and performing any querying or analysis associated with one or more data sets may be stored in the memory device 760. Optionally, the program instructions may be stored on a tangible, non-transitory computer-readable medium such as a compact disk, a digital disk, flash memory, a memory card, a universal serial bus (USB) drive, an optical disc storage medium and/or other recording medium.

An optional display interface 730 may permit information from the bus 710 to be displayed on the display 735 in audio, visual, graphic or alphanumeric format. Communication with external devices may occur using various communication devices and/or ports 740. A communication port 740 may be attached to a communications network, such as the Internet or an intranet. Communication devices may include wireless transceivers for receiving and/or relaying telemetry. That is, a transceiver may comprise both a transmitter and a receiver.

The hardware may also include an interface 745 which allows for receipt of data from input devices such as a keypad 750 or other input device 755 such as a touch screen, a remote control, a pointing device, a video input device and/or an audio input device.

While this disclosure describes example embodiments for example fields and applications, it should be understood that the disclosure is not limited to the disclosed examples. Other embodiments and modifications thereto are possible and are within the scope and spirit of this disclosure. For example, and without limiting the generality of this paragraph, embodiments are not limited to the software, hardware, firmware, and/or entities illustrated in the figures and/or described in this document. Furthermore, embodiments (whether or not explicitly described) have significant utility to fields and applications beyond the examples described in this document.

Embodiments have been described in this document with the aid of functional building blocks illustrating the implementation of specified functions and relationships. The boundaries of these functional building blocks have been arbitrarily defined in this document for the convenience of the description. Alternate boundaries can be defined as long as the specified functions and relationships (or their equivalents) are appropriately performed. Also, alternative embodiments can perform functional blocks, steps, operations, methods, etc. using orderings different than those described in this document.

The features from different embodiments disclosed herein may be freely combined. For example, one or more features from a method embodiment may be combined with any of the system or product embodiments. Similarly, features from a system or product embodiment may be combined with any of the method embodiments herein disclosed.

References in this document to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Furthermore, when a particular feature, structure, or characteristic is described in connection with an embodiment, it would be within the knowledge of persons skilled in the relevant art(s) to incorporate such feature, structure, or characteristic into other embodiments, whether or not explicitly mentioned or described in this document. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but still co-operate or interact with each other.

In this document, “electronic communication” refers to the transmission of data via one or more signals between two or more electronic devices, whether through a wired or wireless network, and whether directly or indirectly via one or more intermediary devices. Devices are “communicatively connected” if the devices are able to send and/or receive data via electronic communication. Any communication unit may include a transmitter and receiver. Telemetry refers to electronic communication of sensor data. Wireless telemetry may use BLUETOOTH LOW ENERGY (BLE) protocols, Zigbee, Wimax, WiFi, near field communications (NFC), tissue conductance communication (TCC), RFID, or other wireless communication protocols. TCC is an intrabody communication protocol which allows implantable devices to communicate with each other.

The invention may be further described by reference to the following numbered clauses:
Clause 1. A telemetry system comprising:
   one or more implants, each of the one or more implants comprising:
      a sensor configured to obtain data related to a subject;
      a distinguishing identifier; and
      a transmitter configured to transmit the distinguishing identifier and the obtained data; and
   a telemetry configuration system configured to:
      display a representation of an anatomical structure of the subject, the representation including a plurality of potential implant positions, each potential implant position being user-selectable; and
      receive a user selection of one of the plurality of potential implant positions;
      receive the distinguishing identifier of a selected implant of the one or more implants;
      in response to receiving an indication that the selected implant has been installed, associate the distinguishing identifier with the user-selected implant position; and
      in response to receiving physical location information related to the installed implant, associate the installed implant with the received location information.
Clause 2. The telemetry system of clause 1, further comprising:
   a remote reader comprising a display device, the remote reader configured to:
      receive the distinguishing identifier and the data obtained from the one or more implants; and
      display the obtained data on the display device.
Clause 3. The telemetry system of clause 2, wherein the telemetry configuration system is configured to associate the selected implant with the user-selected implant position by associating the selected implant with the user-selected implant position in a data store accessible to the remote reader.
Clause 4. The telemetry system of any preceding clause, wherein the telemetry configuration system is configured to receive the distinguishing identifier of the selected implant by wirelessly receiving a unique identifier.
Clause 5. The telemetry system of any preceding clause, wherein the telemetry configuration system is configured to receive the distinguishing identifier of the selected implant by receiving, via a camera, a machine-readable code associated with the selected implant.
Clause 6. The telemetry system of any preceding clause, wherein receiving the indication that the selected implant has been installed comprises:
   receiving sensor data from the selected implant; and
   recognizing that the received sensor data includes a signature that the selected implant has been installed.
Clause 7. The telemetry system of any preceding clause, wherein the representation of the anatomical structure of the subject comprises a medical image of the subject.
Clause 8. The telemetry system of any preceding clause, wherein receiving the physical location information related to the installed implant comprises receiving the physical location information from a surgical navigation system.
Clause 9. The telemetry system of any preceding clause, wherein receiving the physical location information related to the installed implant comprises analyzing a medical image to determine the physical location information.
Clause 10. The telemetry system of any preceding clause, wherein the telemetry configuration system is further configured to, after receiving the user selection of one of the plurality of potential implant positions:
   receive user annotation related to the user-selected implant position; and
   associate the user annotation with the user-selected implant position.
Clause 11. The telemetry system of any preceding clause, wherein the telemetry configuration system is configured to receive the physical location information related to the installed implant by receiving tracking information associated with a tool in contact with the installed implant.
Clause 12. The telemetry system of any preceding clause, wherein receiving the physical location information related to the installed implant comprises:
   receiving a medical image of the subject; and
   receiving a location of the installed implant in the medical image.
Clause 13. The telemetry system of any preceding clause, further comprising a surgical planning system, wherein the telemetry configuration system is configured to receive the distinguishing identifier of the selected implant from the surgical planning system.
Clause 14. A method of configuring a telemetry system, the method comprising:
   displaying a representation of an anatomical structure of a subject, the representation including a plurality of potential implant positions, each potential implant position being user-selectable;
   receiving a user selection of one of the plurality of potential implant positions;
   receiving a distinguishing identifier of a selected implant;
   in response to receiving an indication that the selected implant has been installed, associating the distinguishing identifier with the user-selected implant position; and
   in response to receiving physical location information related to the installed implant, associating the installed implant with the received location information.
Clause 15. The method of clause 14, further comprising, after receiving the user selection of one of the plurality of potential implant positions:
   receiving a user annotation related to the user-selected implant position; and
   associating the user annotation with the user-selected implant position.
Clause 16. The method of any of clauses 14-15, wherein receiving the distinguishing identifier of the selected implant comprises wirelessly receiving a unique identifier from the selected implant.
Clause 17. The method of any of clauses 14-16, wherein receiving the indication that the selected implant has been installed comprises:
   receiving sensor data from the selected implant; and
   recognizing that the received sensor data includes a signature that the selected implant has been installed.
Clause 18. The method of any of clauses 14-17, wherein receiving the physical location information related to the installed implant comprises analyzing a medical image to determine the physical location information.
Clause 19. The method of any of clauses 14-18, wherein:
   the method further comprises receiving a pre-operative medical image of the subject; and
   displaying the representation of the anatomical structure of the subject comprises displaying the received medical image of the subject.
Clause 20. The method of any of clauses 14-19, wherein receiving the indication that the selected implant has been installed comprises receiving the indication from a robotic navigation system.

The breadth and scope of this disclosure should not be limited by any of the above-described example embodiments but should be defined only in accordance with the following claims and their equivalents.

1. A telemetry system comprising:
one or more implants, each of the one or more implants comprising:
a sensor configured to obtain data related to a subject;
a distinguishing identifier; and
a transmitter configured to transmit the distinguishing identifier and the obtained data; and
a telemetry configuration system configured to:
display a representation of an anatomical structure of the subject, the representation including a plurality of potential implant positions, each potential implant position being user-selectable; and
receive a user selection of one of the plurality of potential implant positions;
receive the distinguishing identifier of a selected implant of the one or more implants;
in response to receiving an indication that the selected implant has been installed, associate the distinguishing identifier with the user-selected implant position; and
in response to receiving physical location information related to the installed implant, associate the installed implant with the received location information.

2. The telemetry system of claim 1, further comprising:
a remote reader comprising a display device, the remote reader configured to:
receive the distinguishing identifier and the data obtained from the one or more implants; and
display the obtained data on the display device.

3. The telemetry system of claim 2, wherein the telemetry configuration system is configured to associate the selected implant with the user-selected implant position by associating the selected implant with the user-selected implant position in a data store accessible to the remote reader.

4. The telemetry system of claim 1, wherein the telemetry configuration system is configured to receive the distinguishing identifier of the selected implant by wirelessly receiving a unique identifier.

5. The telemetry system of claim 1, wherein the telemetry configuration system is configured to receive the distinguishing identifier of the selected implant by receiving, via a camera, a machine-readable code associated with the selected implant.

6. The telemetry system of claim 1, wherein receiving the indication that the selected implant has been installed comprises:
receiving sensor data from the selected implant; and
recognizing that the received sensor data includes a signature that the selected implant has been installed.

7. The telemetry system of claim 1, wherein the representation of the anatomical structure of the subject comprises a medical image of the subject.

8. The telemetry system of claim 1, wherein receiving the physical location information related to the installed implant comprises receiving the physical location information from a surgical navigation system.

9. The telemetry system of claim 1, wherein receiving the physical location information related to the installed implant comprises analyzing a medical image to determine the physical location information.

10. The telemetry system of claim 1, wherein the telemetry configuration system is further configured to, after receiving the user selection of one of the plurality of potential implant positions:
receive user annotation related to the user-selected implant position; and
associate the user annotation with the user-selected implant position.

11. The telemetry system of claim 1, wherein the telemetry configuration system is configured to receive the physical location information related to the installed implant by receiving tracking information associated with a tool in contact with the installed implant.

12. The telemetry system of claim 1, wherein receiving the physical location information related to the installed implant comprises:
receiving a medical image of the subject; and
receiving a location of the installed implant in the medical image.

13. The telemetry system of claim 1, further comprising a surgical planning system, wherein the telemetry configuration system is configured to receive the distinguishing identifier of the selected implant from the surgical planning system.

14. A method of configuring a telemetry system, the method comprising:
displaying a representation of an anatomical structure of a subject, the representation including a plurality of potential implant positions, each potential implant position being user-selectable;
receiving a user selection of one of the plurality of potential implant positions;
receiving a distinguishing identifier of a selected implant;
in response to receiving an indication that the selected implant has been installed, associating the distinguishing identifier with the user-selected implant position; and
in response to receiving physical location information related to the installed implant, associating the installed implant with the received location information.

15. The method of claim 14, further comprising, after receiving the user selection of one of the plurality of potential implant positions:
receiving a user annotation related to the user-selected implant position; and
associating the user annotation with the user-selected implant position.

16. The method of claim 14, wherein receiving the distinguishing identifier of the selected implant comprises wirelessly receiving a unique identifier from the selected implant.

17. The method of claim 14, wherein receiving the indication that the selected implant has been installed comprises:
receiving sensor data from the selected implant; and
recognizing that the received sensor data includes a signature that the selected implant has been installed.

18. The method of claim 14, wherein receiving the physical location information related to the installed implant comprises analyzing a medical image to determine the physical location information.

19. The method of claim 14, wherein:
the method further comprises receiving a pre-operative medical image of the subject; and
displaying the representation of the anatomical structure of the subject comprises displaying the received medical image of the subject.

20. The method of claim 14, wherein receiving the indication that the selected implant has been installed comprises receiving the indication from a robotic navigation system.
